(19) United States
(12) Patent Application Publication
(10) Pub. No.: US 2024/0350746 A1
(43) Pub. Date: Oct. 24, 2024

(54) System, Apparatus, and Method for Delivering a Drug into a Target Volume of a Subject

(71) Applicant: NESA MEDTECH PRIVATE LIMITED, Bangalore (IN)

(72) Inventors: Arun Paliakkara JOSE, Thrissur (IN); Suraj Rajan Kanyan KANDI, Pune (IN); Prajna Umanatha SHETTY, Udupi (IN); Sanjay GIRISH, Bangalore (IN)

(21) Appl. No.: 18/683,443

(22) PCT Filed: Aug. 12, 2022
(86) PCT No.: PCT/IN2022/050729

§ 371 Date: Feb. 13, 2024

(30) Foreign Application Priority Data
Aug. 13, 2021 (IN) 202141034052

(51) Int. Cl. A61M 5/42; A61M 5/24; A61M 5/31; A61M 5/315; A61M 25/01; A61M 25/06

(52) U.S. Cl. CPC A61M 5/427; A61M 5/2422; A61M 5/3129; A61M 5/31505; A61M 5/31551; A61M 25/0662; A61M 2025/0166; A61M 2205/3375; A61M 2205/502; A61M 2205/583; A61M 2210/1433

(57) ABSTRACT

A system, apparatus, and method for delivering a drug into a target volume of a subject includes an ultrasound scanner for performing a volume scan of a predetermined volume of the subject, a computing device for generating a 3D model of the predetermined volume and computing navigational data to reach the target volume from an entry orifice of the volume of the subject, a housing, for accommodating a barrel, to which a sheath is detachably attached, and a drug delivery device for delivering the drug at one or more locations in the target volume, wherein the drug delivery device is configured for delivering a predetermined volume of drug at one or more locations in the target volume of the subject.

### TECHNICAL FIELD

The present disclosure generally relates to the field of electro-mechanical medical devices and systems, and more particularly to a system, apparatus, and method for delivering drug into a target volume of a subject.

### BACKGROUND

Fibroids, tumors, cysts, and similar growths are found in the body, and gaining accesses and administering treatment to the precise location with precise orientation in these areas is a challenge. With the present technologies, the surgeons still find it challenging to administer the treatment with precision at the region. But with the present advancement in imaging systems combined with the right software and hardware combination, reaching the desired location may be achieved making it much simpler for the surgeons to use such system for more surgical procedures. In the present case a uterine fibroid is taken as an example and the system apparatus and method can be extended to multiple surgical treatments.

Uterine fibroids are generally non-cancerous tumors in the uterus and rarely turn out to be cancerous. Uterine fibroids are one of the most common gynecological disorders. Currently, there are no definitive ways of diagnosis. Imaging and tracking over a period of few months along with a few tests is the most reliable way. The definitive way, however, is to surgically resect it and then subject it to pathological analysis and study to make a diagnosis. The first line of treatment is management of symptoms using medication. However, this approach is often not effective because of the challenges in administering the medications to the precise locations in the uterus. Uterine fibroids, when symptomatic, require surgical treatment and the most common surgical treatment is the removal of the uterus, called hysterectomy. Such a treatment may result in high morbidity, long recovery time, and cause damages to the organs in the proximity of the uterus.

Another minimally invasive surgical option is the removal of fibroids through laparoscopic procedures or through hysteroscopic procedures. Other procedures include magnetic resonance guided focused ultrasound and uterine artery embolization. However, these procedures require expensive infrastructure as well as skilled clinicians such as interventional radiologists. Yet another alternative is the use of ablation methods such as radio frequency ablation, microwave ablation, cryogenic ablation, laser ablation, and plasma ablation. Ablation is carried out by passing energy precisely into the target location and ablating the fibroid under the guidance of ultrasound imaging. In radio frequency ablation, necrosis is induced in the fibroids by thermal necrosis, with an electrode, for example.

Most of the above-mentioned surgical treatment procedures require general anesthesia and highly skilled clinicians as it is difficult to provide the therapy with precision and constrain it in the required space without causing harm to neighboring tissues of the uterus.

In addition to the surgical treatment, significant advancements in medical research and development have been made to treat uterine fibroids with drugs. However, the use of drugs is limited due to unavailability of precise drug delivery devices which may deliver drug precisely into a desired or predetermined location in the uterus, including fibroids, directly rather than orally.

Even though biopsy is effective, as the organ is below the urinary bladder and has limited access, biopsies often lead to inaccurate piercing leading to acquiring samples from unintended locations. It is also difficult to acquire the sample safely and effectively for the biopsy. This may also result in a false negative result and misdiagnosis. Even though different types of guidance technologies have been adopted for biopsy, there is a need for a device to guide the biopsy needles to the right position.

In addition to the procedures described above procedures such as laparoscopic and hysteroscopic surgeries require highly skilled clinicians as it is difficult to reach the desired locations and hence difficult to provide the therapy with precision.

### BRIEF SUMMARY

This summary is provided to introduce a selection of concepts in a simple manner that is further described in the detailed description of the disclosure. This summary is not intended to identify key or essential inventive concepts of the subject matter nor is it intended for determining the scope of the disclosure.

To overcome at least one of the problems mentioned above, there exists a need for a system, apparatus, and method for delivering a drug to one or more locations in a target volume of a subject.

The present disclosure discloses a system for delivering a drug into a target volume of a subject, the target volume may include fibroids, tumors, cysts, and similar growths are found in the body. The system comprises, an ultrasound scanner configured for performing a volume scan of a predetermined volume of the subject, wherein the scanned predetermined volume includes the target volume, a computing device, communicatively connected to the ultrasound scanner, the computing device configured for at least generating a three-dimensional model of the predetermined volume and computing navigational data to reach the target volume from an entry orifice of the volume of the subject, a housing unit, for accommodating a barrel, to which a sheath is detachably attached, and a drug delivery device for delivering the drug at one or more locations in the target volume, wherein the drug delivery device comprises, a hollow cylindrical body comprising a needle at a first end, the hollow cylindrical body configured for storing the drug to be delivered and configured for moving the needle linearly through the sheath to pierce the target volume and to reach the one or more locations in the target volume, and a plunger, positioned inside the hollow cylindrical body, wherein the plunger is configured for enabling delivery of a predetermined volume of a drug to the one or more locations in the target volume.

To further clarify advantages and features of the present disclosure, a more particular description of the disclosure will be rendered by reference to specific embodiments thereof, which is illustrated in the appended figures. It is to be appreciated that these figures depict only typical embodiments of the disclosure and are therefore not to be considered limiting of its scope. The disclosure will be described and explained with additional specificity and detail with the accompanying figures.

### BRIEF DESCRIPTION OF THE FIGURES

The disclosed method and system will be described and explained with additional specificity and detail with the accompanying figures in which:

FIG. 1 illustrates an exemplary system for delivering a drug into a target volume of a subject in accordance with an embodiment of the present disclosure;

FIG. 2 is a block diagram of an exemplary computing device in accordance with an embodiment of the present disclosure;

FIG. 3A & 3B shows an arrangement of the drug delivery device in accordance with an embodiment of the present disclosure;

FIG. 4A shows an exemplary sheath in accordance with an embodiment of the present disclosure;

FIG. 4B shows an exemplary locking mechanism for attaching the sheath to the housing unit;

FIG. 4C shows an exemplary connector in accordance with an embodiment of the present disclosure;

FIG. 5 shows the housing unit in accordance with an embodiment of the present disclosure;

FIG. 6A and 6B show the barrel connected to the housing unit in accordance with an embodiment of the present disclosure;

FIG. 7 shows an exemplary drug delivery device in accordance with an embodiment of the present disclosure;

FIG. 8 shows an exemplary arrangement of the drug delivery device and the barrel in accordance with an embodiment of the present disclosure;

FIG. 9A and 9B shows an alternative mechanism for moving the needle to the one or more location in the target volume in accordance with an embodiment of the present disclosure;

FIG. 9C shows arrangement of the alternative mechanism with the barrel and the housing in accordance with an embodiment of the present disclosure;

FIG. 10A shows an exemplary plunger in accordance with an embodiment of the present disclosure;

FIG. 10B shows the front view of the knob of the barrel with marking and graduated knob of the plunger in accordance with an embodiment of the present disclosure;

FIG. 10C shows another exemplary plunger in accordance with an embodiment of the present disclosure;

FIG. 11 illustrates the one or more location determined by the computing device in accordance with an embodiment of the present disclosure;

FIG. 12 shows exemplary feedback displayed by the computing device in accordance with an embodiment of the present disclosure;

FIG. 13 shows sheaths with different bent profiles; and

FIG. 14 shows an exemplary pivotable sheath with pivotable profiles in accordance with an embodiment of the present disclosure.

Further, persons skilled in the art to which this disclosure belongs will appreciate that elements in the figures are illustrated for simplicity and may not have been necessarily drawn to scale. Furthermore, in terms of the construction of the joining ring and one or more components of the bearing assembly may have been represented in the figures by conventional symbols, and the figures may show only those specific details that are pertinent to understanding the embodiments of the present disclosure so as not to obscure the figures with details that will be readily apparent to those of ordinary skill in the art having benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications to the disclosure, and such further applications of the principles of the disclosure as described herein being contemplated as would normally occur to one skilled in the art to which the disclosure relates are deemed to be a part of this disclosure.

It will be understood by those skilled in the art that the foregoing general description and the following detailed description are exemplary and explanatory of the disclosure and are not intended to be restrictive thereof.

In the present disclosure, relational terms such as first and second, and the like, may be used to distinguish one entity from the other, without necessarily implying any actual relationship or order between such entities.

The terms “comprises”, “comprising”, or any other variations thereof, are intended to cover a non-exclusive inclusion, such that a process or method that comprises a list of steps does not include only those steps but may include other steps not expressly listed or inherent to such a process or a method. Similarly, one or more elements or structures or components preceded by “comprises . . . a” does not, without more constraints, preclude the existence of other elements, other structures, other components, additional devices, additional elements, additional structures, or additional components. Appearances of the phrase “in an embodiment”, “in another embodiment” and similar language throughout this specification may, but do not necessarily, all refer to the same embodiment.

Unless otherwise defined, all technical and scientific terms used herein have the same meaning as commonly understood by one of ordinary skill in the art to which this disclosure belongs. The components, methods, and examples provided herein are illustrative only and not intended to be limiting.

Embodiments of the present disclosure will be described below in detail with reference to the accompanying figures.

Embodiments of the present disclosure disclose a system, apparatus, and method for delivering a drug into a target volume of a subject. The system comprises an ultrasound scanner for performing a volume scan of a predetermined volume of the subject, a computing device configured for generating a three-dimensional model of the predetermined volume and computing navigational data to reach the target volume from an entry orifice of the volume of the subject, a housing, for accommodating a barrel, to which a sheath is detachably attached, and a drug delivery device for delivering the drug at one or more locations in the target volume. The drug delivery device comprises, a hollow cylindrical body comprising a needle at a first end, the hollow cylindrical body configured for storing the drug to be delivered and configured for moving the needle linearly through the sheath to pierce the target volume and to reach the one or more locations in the target volume, and a plunger, positioned inside the hollow cylindrical body, wherein the plunger is configured for enabling delivery of a predetermined volume of a drug to the one or more locations in the target volume.

The term “target volume” as described herein refers to fibroids, tumors, cysts, or similar growths that are found in the body of a subject and the term “one or more locations in the target volume” refers to one or more locations in fibroids, tumors, cysts, or similar growths where a predetermined volume of drug needs to be delivered for treatment. Alternatively, Further, the “drug” may be in one of a liquid form, a capsule form, or a powder form.

The term “predetermined volume” of the subject as described herein refers to a volume of the body that a user scans to identify the target region. That is, the predetermined volume is a volume surrounding the target volume. For example, considering fibroid as a target volume, a uterus of the subject is the predetermined volume. It is to be noted that the user may be a physician, a surgeon, or any healthcare professional. Herein after the term “user” is used in the present disclosure.

For the ease of explanation and understanding, the method and the working mechanism of the system and apparatus is described considering a “fibroid” as the target volume and a “uterus” of the subject as the predetermined volume. However, the system and apparatus can be used for treating tumors, cysts, or similar growths that are found in the body of the subject, with minor or no modification to the system, the apparatus, and the method. For example, the disclosed system, apparatus, and method may be used for procedures such as laparoscopic and hysteroscopic surgeries, which are done through a natural orifice or an artificial orifice.

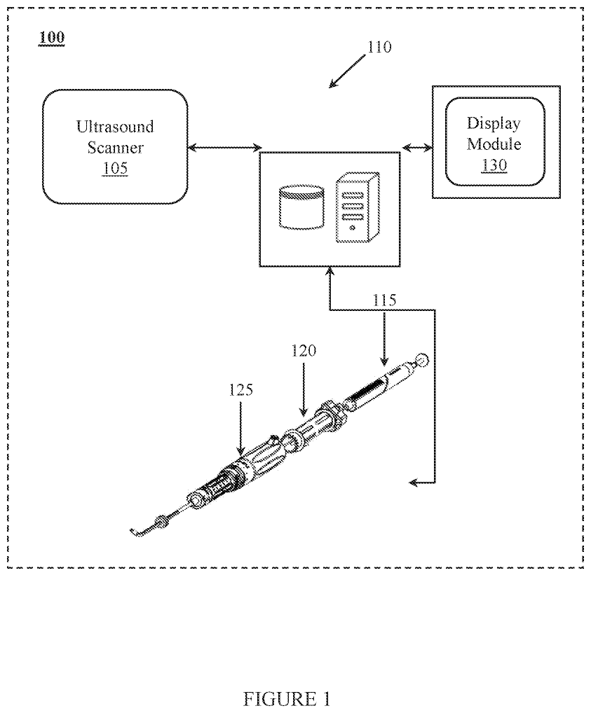

FIG. 1 illustrates an exemplary system 100 for delivering a drug into a target volume of a subject in accordance with an embodiment of the present disclosure. As shown, the system 100 comprises an ultrasound scanner 105, a computing device 110, and a drug delivery device 115, wherein the drug delivery device 115 is housed inside a barrel 120, and the barrel 120 is housed on a housing unit 125. It is to be noted that the ultrasound scanner 105 and the computing device 110 are communicatively connected through any known wired or wireless communication network (not shown).

The ultrasound scanner 105 may be any known type of ultrasound scanner, with an ultrasound transducer (not shown), used to capture live images. In the present system 100, the ultrasound scanner 105 is used for performing a volume scan of a predetermined volume of the subject, wherein the scanned predetermined volume includes the target volume. For example, while treating fibroids, the ultrasound transducer is inserted through an external orifice of the uterus so that the distal end of the transducer reaches the cervix. Here the term distal and proximal are with reference to the user or other health care provider delivering the treatment and the words are used consistently with the same meaning hereinafter, unless otherwise stated. For the sake clarity, it is to be noted that the proximal end of the transducer has a cable for connecting to the ultrasound scanner 105 and the ultrasound transducer positioned on the cervix by the user. The distal end of the ultrasound transducer is then swept along the uterus to acquire a volume scan of the entire uterus (the predetermined volume) in different orientations. The resultant images are captured by the ultrasound scanner 105 and communicated to the computing device 110.

The computing device 110 may include, for example, a computer, a mainframe computer, a computer server, or a network of computers which provides functionalities and services to other programs or devices such as the ultrasound scanner 105 and the drug delivery device 115. In one embodiment of the present disclosure, the computing device 110 is configured for generating a three-dimensional model (3D model) of the predetermined volume and computing navigational data to reach the target volume from an entry orifice of the volume of the subject.

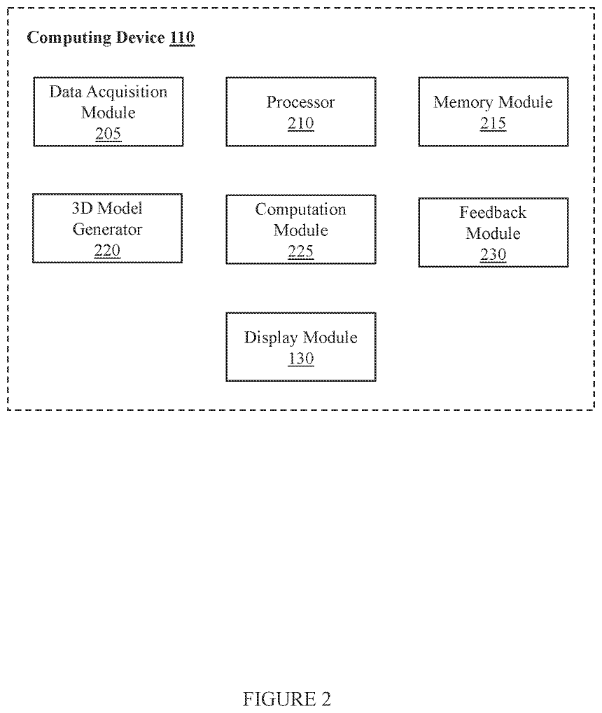

FIG. 2 is a block diagram of an exemplary computing device 110 in accordance with an embodiment of the present disclosure. As shown, the computing device 110 comprises a data acquisition module 205, processor 210, a memory module 215, a 3D model generator 220, a computation module 225, a feedback module 230 and the display module 130. The memory module 215 stores the instructions to be processed by the processor and intermediate data during the processing.

Initially, the data acquisition module 205 acquires the volume scan images from the ultrasound scanner 105 and records in the memory module 215. In one embodiment, the data acquisition module 205 displays the images on the display module 130 for providing feedback to the user on the scan coverage during the scan. Further, acquisition module 205 displays the one or more images (frames) for annotations by the user. It is to be noted that the user may select one or more images for annotations. In one embodiment, an interface is provided with one or more markers (colours, for example) to highlight the target volume, the predetermined volume, and other locations in the predetermined volume, if required. In another embodiment of the present disclosure, a machine learning model is trained to identify the target volume in the images of the predetermined volume. The machine learning model is trained using the target volume identified by the user. Once this is done, the system may recommend the target volume to the user for consideration. The user may ignore the suggestion and determine the target volume and annotate the same. On receiving the user annotations, the computation module 225 computes the navigational data to reach the target volume from an entry orifice of the predetermined volume of the subject. Further, the 3D model generator 220 generates a 3D model of the predetermined volume of the subject, the predetermined volume including the target volume. Considering the example of fibroids, on receiving the images of the uterus from the ultrasound scanner 105, the computing device 110 displays the one or more images for annotation by the user. The user may use one or more markers (colours, for example) to highlight the fibroids, the uterus, and other locations of the uterus, if required. On receiving the user annotations, the computation module 225 computes the navigational data to reach the fibroids from the orifice of the uterus. Further, the 3D model generator 220 generates a 3D model of the uterus. It is to be noted that the 3D model of the uterus is generated using a plurality of 2D images by any know methods. The generated 3D model is displayed on the display module 130 for providing feedback to the user.

As described, on receiving the annotations from the user, the computation module 225 computes the navigational data to reach the target volume from an entry orifice of the predetermined volume of the subject. In one embodiment, the 3D model is displayed with navigational parameters on the display module 130 to assist the user during the procedure. In one embodiment, the navigational data include but not limited to type of a sheath, length of insertion of sheath into the predetermined volume, orientation of the sheath, length of a needle to reach the one or more locations of the target volume, and angles of rotation of a graduated knob (of the drug delivery device 115) for delivering the drug at each of the one or more locations in the target volume. Further, in one embodiment of the present disclosure, the computation module 225 is configured for determining one or more locations in the target volume where the drug needs to be delivered. It is to be noted that the one or more locations as described herein may include a center of the target volume or any other locations in the target volume, and the locations are determined based on at least volume (size) of the target volume, a shape of the target volume, and a location of the target volume in the predetermined volume. Furthermore, the computation module 225 is configured for computing a volume of the drug to be delivered at the one or more locations of the target volume, wherein the volume of drug is computed based at least one of a volume of the target volume, a shape of the target volume, a location of the target volume in the predetermined volume. Such information is displayed on the display module 130 for providing feedback, in other words, for assisting the user in drug delivery procedure. It is to be noted that the feedback may be provided by displaying the length of insertion of the needle or the number of rotations or an angle of rotation of the barrel, for piercing the target volume and to reach the one or more location. Similarly, the feedback may be provided by displaying the length of the needle or the number of rotations or an angle of rotation of the plunger for delivering the volume of drug to be delivered at each of the one or more locations. The user may ignore the suggestion and determine the volume of drug to be delivered based on the clinically presented condition of the subject, the user's personal judgement, any changes in the protocol for the drug not pre-programmed into the computing device 110 or accept at the user's description. In addition, since the volume of drug to be delivered depends on the concentration and effectiveness of the drug on the target region, the computation module 225 considers the effectiveness of the drug on the target region (like fibroid, cyst, polyps, etc.) and the volume of the target volume, a shape of the target volume, location of the target volume in the predetermined volume, the protocol published for the drug, etc., while computing and recommending the volume of the drug to be delivered to the one or more locations in the target volume. The user may ignore the suggestion and determine the volume of drug to be delivered based on the clinically presented condition of the subject, the user's personal judgement, any changes in the protocol for the drug not pre-programmed into the computing device 110 or accept at the surgeon's description. In one embodiment of the present disclosure, a machine learning model is trained to determine the one or more locations in the target volume. For example, a plurality of images of the predetermined volume marked with target volume, preferred locations for drug delivery, etc., may be used for training the model. Then for any new image, the model is used for determining the one or more locations for delivering the drug. The way the computing device 110 computes and provides the navigational data is described in detail further below, considering the example of uterus fibroids.

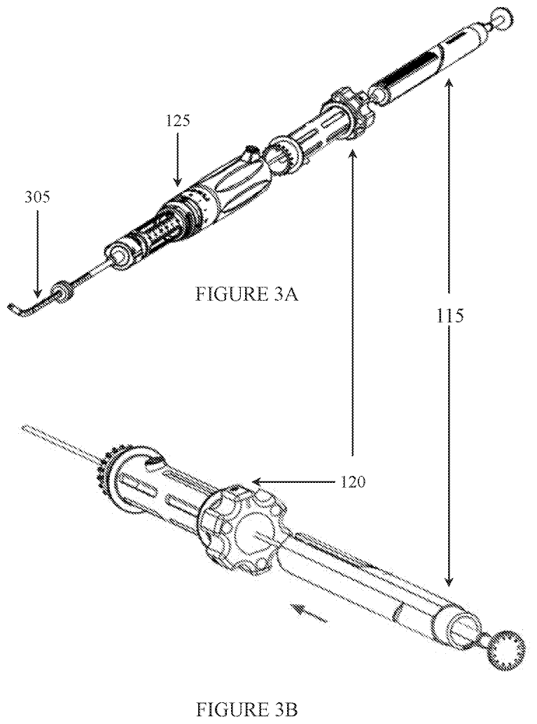

FIG. 3A & 3B shows an arrangement of the drug delivery device 115 in accordance with an embodiment of the present disclosure. As shown, the drug delivery device 115 is arranged inside the barrel 120, wherein the barrel 120 is positioned in the housing unit 125. Further, the housing unit 125 comprises a sheath 305 at a distal end (end away from a user of the drug delivery device 115), wherein the sheath 305 is detachably attached to the housing unit 125. It is to be noted that the different sheaths having different bend angles may be used depending on the location of the target volume inside the predetermined volume of the subject. In one embodiment, the angles of the sheath 305 may vary from 0 degrees to 45 degrees. As described, the computing device 110 suggest the type of the sheath to be used based on the location of the target volume. In one embodiment of the present disclosure, the drug delivery device 105, the barrel 120 and the housing unit 125 are arranged in such a way that a rotation of the barrel 120 moves the drug delivery device 115 linearly for moving a needle of the drug delivery device 115, through a sheath of the housing unit 125, for piercing the target volume and to reach the one or more locations inside the target volume.

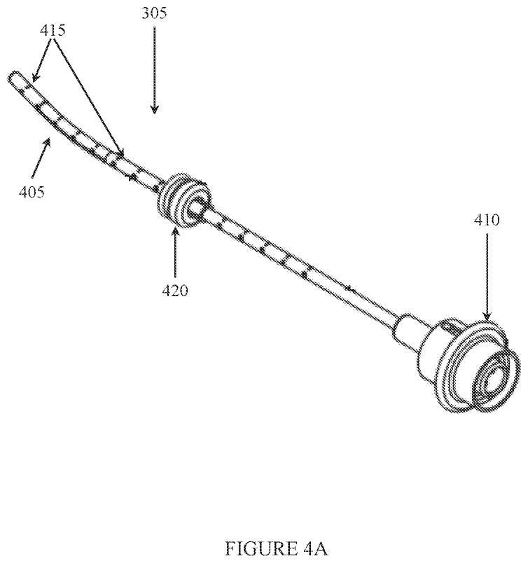

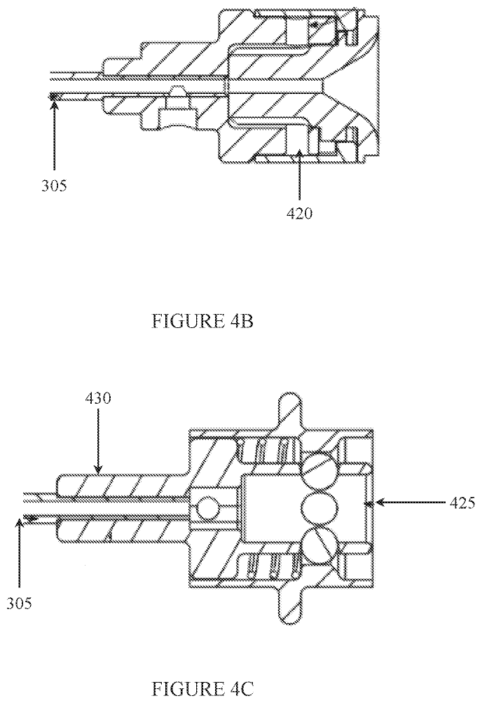

FIG. 4A shows an exemplary sheath 305 in accordance with an embodiment of the present disclosure. As shown, a distal end 405 (end away from the housing unit 125) of the sheath 305 is bent at a predefined angle for reaching the one or more locations in the target volume. The proximal end comprises a locking mechanism 410 to connect to the housing unit 125. In one embodiment of the present disclosure, the sheath 305 comprises markings 415 for providing visual feedback to the user on the insertion depth of the sheath 305 in the uterine cavity. Further, a stopper 420 is provided on the sheath 305, wherein the stopper 420 is adjustable on the sheath 305 based on the required length to prevent accidental insertion of the sheath 305 into the uterus. As described, the required length (the insertion length) is computed by the computation module 225. Furthermore, the sheath 305, specifically the bent portion of the sheath 305, is designed for the optimum angle to easily reach the walls of the uterine cavity and to support the needle of the drug delivery device 115 to penetrate the target volume of the subject, for example fibroid or any desired region in the uterus. In another embodiment, the sheath is pivotable and may be adjusted to various positions. The round tip at the distal end of the sheath 305 avoids causing any harm to the surrounding tissue as the sheath 305 advances in the uterine cavity.

As described, the sheath 305 is detachably attached to the housing unit 125 through the locking mechanism 420 provided at the base of the sheath 305. FIG. 4B shows an exemplary locking mechanism for attaching the sheath to the housing unit 125. As shown, the sheath 305 may be attached to the housing unit 125 with a rotate and lock feature. The lock mechanism 420 is designed in such a way that the sheath 305 may be assembled only in one position. This feature ensures that the markings 415 on the sheath 305 is always visible to the user to provide visual feedback to the user on the exact position based on the bent angle of the sheath 305 with reference to the user.

In another embodiment, the sheath 305 may be connected to the housing unit 125 using a connector. FIG. 4C shows an exemplary connector in accordance with an embodiment of the present disclosure. As shown, the connector comprises a plug 425 attachable to housing unit 125 and a female socket 430 is fixed to the sheath 305. The female socket 430 comprises an anti-rotation cross section for preventing the sheath 305 from rotating on the housing unit 125. In another embodiment, bayonet mounting may be used to lock the sheath 305 to the housing unit 125. It is to be noted that the markings are provided on the sheath 305 to align the sheath 305 with the housing unit 125. In one embodiment, the sheath 305 comprises a ball plunger mechanism or locks of similar mechanism for loading different sheaths. A person skilled in the art may use other implementations to achieve the same results.

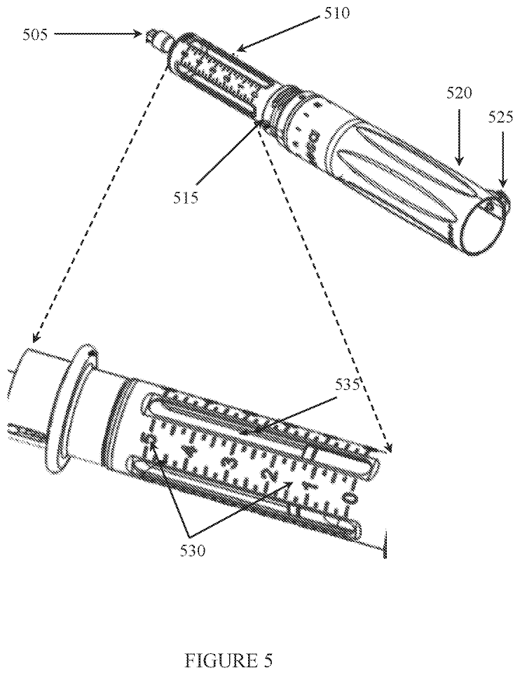

In one embodiment of the present disclosure, the housing unit 125 comprises mechanism for holding the sheath 305 and allow the user to hold and use the drug delivery device 115. FIG. 5 shows the housing unit 125 in accordance with an embodiment of the present disclosure. As shown, the housing unit 125 comprises a housing front end 505, a housing front 510, an actuation switch 515, a housing back 520 and a barrel locking pin 525. In one embodiment of the present disclosure, the barrel locking pin 525 engages with a circular groove on the outer surface of the barrel 120 and locks the barrel 120 in place and only prevents the transitional movement and allows the rotational movement of the barrel 120 with reference to the housing unit 125.

The housing front end 505 comprises a sheath locking features for positioning and locking the sheath 305. The locking feature aligns the sheath 305 in a particular orientation to provide feedback to the user on the orientation of the tip of the sheath 305. The locking feature in the sheath 305 also comprises a taper that guides the drug delivery device 115 during assembly. As described, the rotation of the barrel 120 moves the drug delivery device 115 linearly for moving a needle of the drug delivery device 115, through the sheath of the housing unit 125, for piercing the target volume and to reach the one or more locations inside the target volume. In one embodiment of the present disclosure, needle movement markings 530 are provided on the housing front 510 for indicating the position of the needle as the needle enters the uterine cavity. In one implementation, the markings are made in increments of 2.5 mm, however, they may be 1 mm or higher. Further, cuts 535 are provided on four sides of the housing front 510 to ensure that the needle movement markings 530 are visible in any orientation.

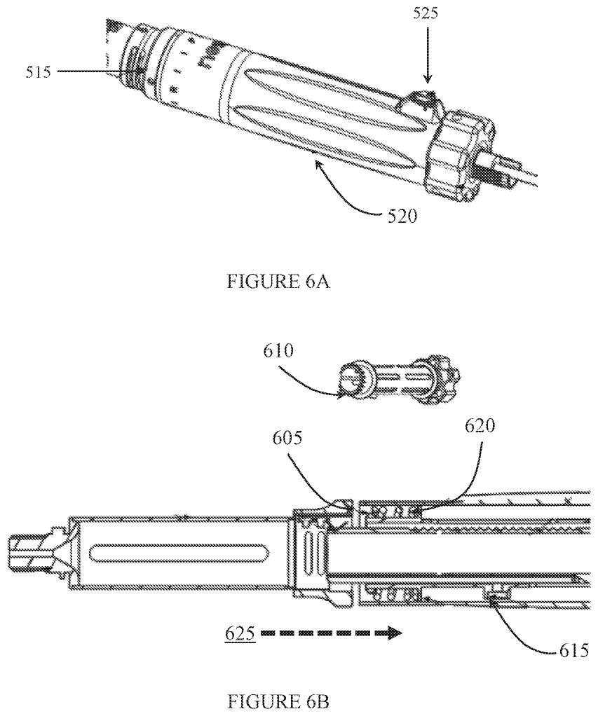

As described, the housing front end 505 connects the sheath 305 to the housing unit 125. The actuation switch 515 (a pullback mechanism) on the housing unit 125 locks the barrel 120 with the housing unit 125. FIG. 6A and 6B show the barrel connected to the housing unit 125 in accordance with an embodiment of the present disclosure. The actuation switch 515 enables rotation of the barrel 210 and advancement of the drug delivery device 115, which is positioned inside the barrel 120, when the barrel 120 is rotated by the user. The actuation switch 515 locks the barrel 120 in position and prevents any unintentional movement of the drug delivery device 115 during the delivery process. As shown, a gear tooth 605 of the actuation switch 515 gets engaged with a gear tooth 610 on the barrel 120 providing a positive lock in the system. When assembled with a spring 620 and the barrel 120, the spring 620 and a pin 615 (on the inner surface of the barrel 120) positions the actuation switch 515 in a position to lock the barrel 120 in position. That is, when the spring 620 is in expanded form (in normal condition), the gear tooth 605 of the actuation switch 515 will be in engagement position with a gear tooth 610 on the barrel 120, thereby restricting linear motion of the drug delivery device 115. During the process, the user pulls the actuation switch 515, in a direction shown by the dotted arrow 625, to compress the spring 620 and this disengages the gear tooth 605 and 610, and rotary motion of the barrel 120 linearly moves the drug delivery 115 for moving the needle toward the target volume. It is to be noted that the pin 615 engages with a helical groove on the outer surface of the drug delivery device 115 enables the linear motion of the drug delivery device 115. It is to be noted that the sheath 305, the barrel 120 and the housing 125 and other parts of these elements may be made of metals such as stainless steel or titanium or metals having properties similar to that of steel and titanium. Alternatively, they may be made of suitable polymers.

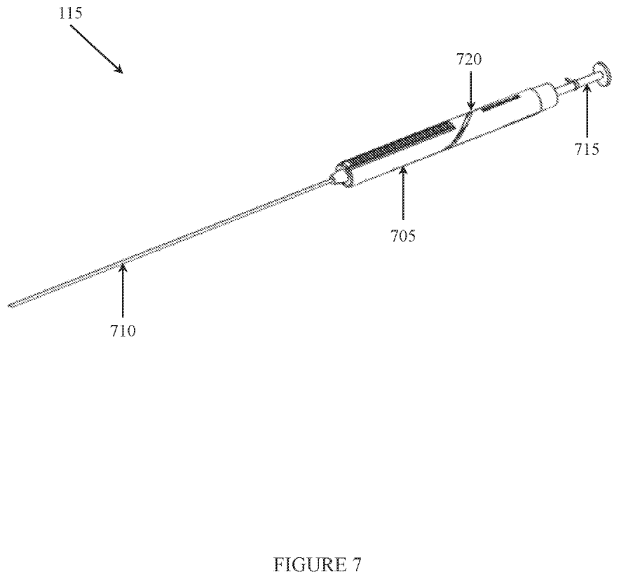

FIG. 7 shows an exemplary drug delivery device 115 in accordance with an embodiment of the present disclosure. As shown, the drug delivery device 115 comprises a hollow cylindrical body 705, a needle 710, and a plunger 715, wherein the plunger 715 is positioned inside the hollow cylindrical body 705.

As shown, the hollow cylindrical body 705 comprises the needle 710 at a first end (end away from the user), wherein the needle 710 is detachably attached to the hollow cylindrica body 705. It is to be noted that the length of the needle 710 may vary between 10 cm to 30 cm. for example. Further, the needle 710 is made up of flexible material having enough strength to pierce the target volume, for example fibroids. In other words, the material, and the configuration of the needle 710 is such that the bend does not impede the flow of the drug and at the same time the needle 710 is strong enough to penetrate the target region. In a preferred embodiment, the needle 710 is made of flexible steel or material having similar properties of flexible steel. The drug is loaded into the hollow cylindrical body 705 of the drug delivery device 115 through the needle tip. In one embodiment of the present disclosure, the hollow cylindrical body 705 is configured for storing the drug to be delivered and further configured for moving the needle 710 linearly through the sheath 305 to pierce the target volume and to reach the one or more locations in the target volume. In one embodiment of the present disclosure, to move the needle 710 linearly through the sheath 305, the hollow cylindrical body 705 comprises a helical groove 720 on an outer surface, wherein the helical groove 720 engages with a pin 615 on the inner surface of the barrel 120, and a rotation of the barrel 120 moves the drug delivery device 115 linearly for moving the needle 710 of the drug delivery device 115, through the sheath 305, for piercing the target volume and to reach the one or more locations inside the target volume.

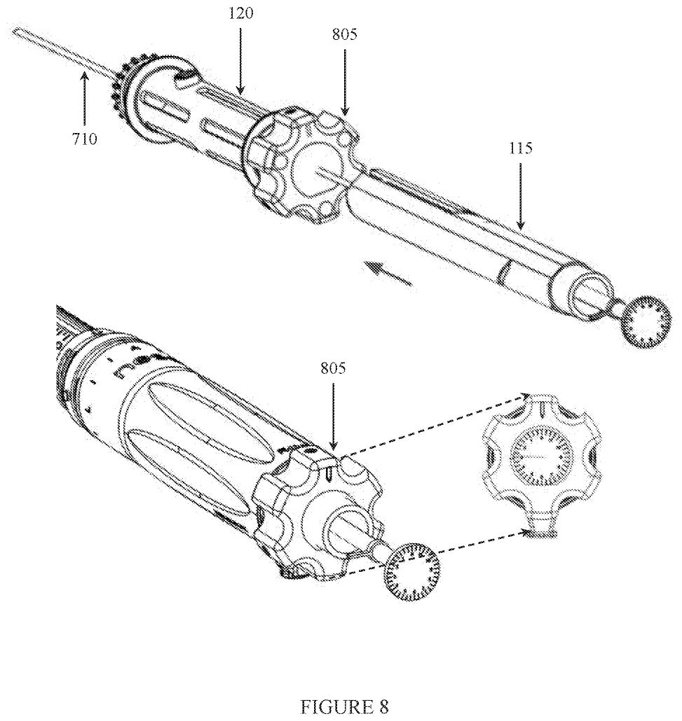

FIG. 8 shows an exemplary arrangement of the drug delivery device and the barrel in accordance with an embodiment of the present disclosure. As shown, when the drug delivery device 115 is arranged inside the barrel 120, the helical groove 720 on the drug delivery device 115 engages with the pin 615 on the inner surface of the barrel 120. In one embodiment of the present disclosure, the barrel 120 comprises a knob 805 which enables rotation of the barrel 120 for moving the drug delivery device 115 linearly. That is, when the user pulls actuation switch 515 and rotates the knob 805 in clockwise direction, the helical groove 720 and the pin 615 moves the drug delivery device 115 linearly and hence the needle 710 moves through the sheath 305 to pierce the target volume to deliver the drug at the one or more locations in the target volume.

In one embodiment of the present disclosure, the drug delivery device 115, the barrel 120 and the housing unit 125 are arranged in such a way that a rotation of the barrel 120 moves the drug delivery device 115 linearly for moving the needle 710 of the drug delivery device 115, through a sheath 305, for piercing the target volume and to reach the one or more locations inside the target volume.

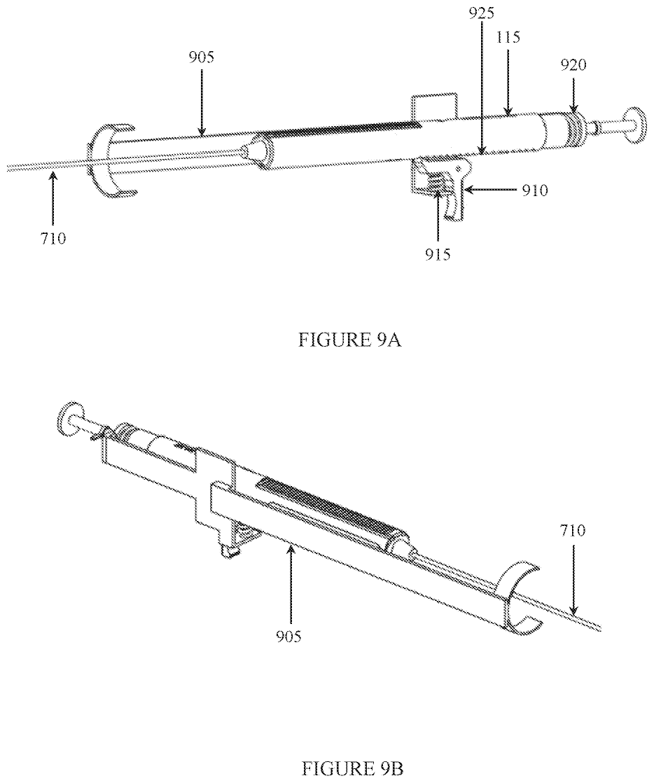

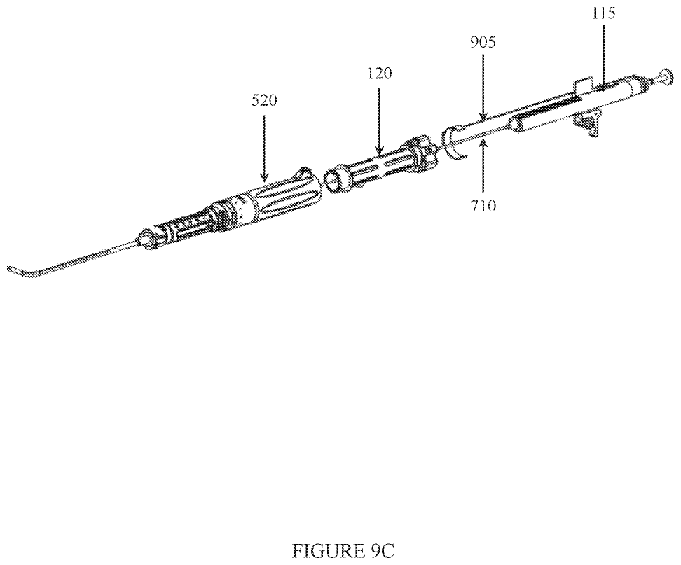

FIG. 9A and 9B shows an alternative mechanism for moving the needle to the one or more location in the target volume in accordance with an embodiment of the present disclosure. FIG. 9C shows arrangement of the alternative mechanism with the barrel and the housing in accordance with an embodiment of the present disclosure. Referring to FIGS. 9A, 9B and 9C, the mechanism comprises holder 905 for holding the drug delivery device 115 and the barrel 120, with housing back 520, and for providing support to the mechanism, a trigger 910 with a first spring 915 in expanded position, and a second spring 920 at the proximal end (the end near to the user) of the drug delivery device 115, wherein the second spring 920 will be in a compressed state, when note used, between the proximal end of the drug delivery device 115 and the proximal end of the barrel 120. In this mechanism, the hollow cylindrical body of the drug delivery device 115 comprises a plurality of teeth 925 perpendicular to the axis of the drug delivery device 115, and the tip of the trigger 910 will be engaged with the one of the plurality of teeth 925 with the action of the first spring 915.

When the trigger 910 is pulled towards the user, the tip of the trigger 910 leans down and gets disengaged from the teeth with which it is engaged. The tension in the second spring 920 at the proximal end of the drug delivery device 115 makes the drug delivery device 115 move in the forward direction thereby moving the needle 715 attached to pierce the target volume and to reach the one or more locations in the target volume. When the trigger 910 is released, the first spring 915 pushes back the trigger 910 to its normal position and the tip of the trigger 910 gets engaged with one of the plurality of teeth 925 on the hollow cylindrical body drug delivery device 115 and restrict further movement of the needle 710 and avoids the backward movement of the needle 710. The movement of the needle 710 is in incremental as the trigger 910 gets disengage from previous teeth and engages to the next teeth and the cycles continue till the needle reaches the required location in the target volume.

As described, based on at least the volume of the target volume and the shape of the target volume, the computing device 110 determines the one or more locations in the target volume for delivering the drug. Further, based on the target volume and the one or more locations, the computing device 110 computes the volume of drug to be delivered at each of the one or more locations in the target volume, and suggest the same to user by displaying n the display module 130. In one embodiment of the present disclosure, the drug delivery device 115 is configured for delivering a predetermined volume of the drug at the one or more locations in the target volume.

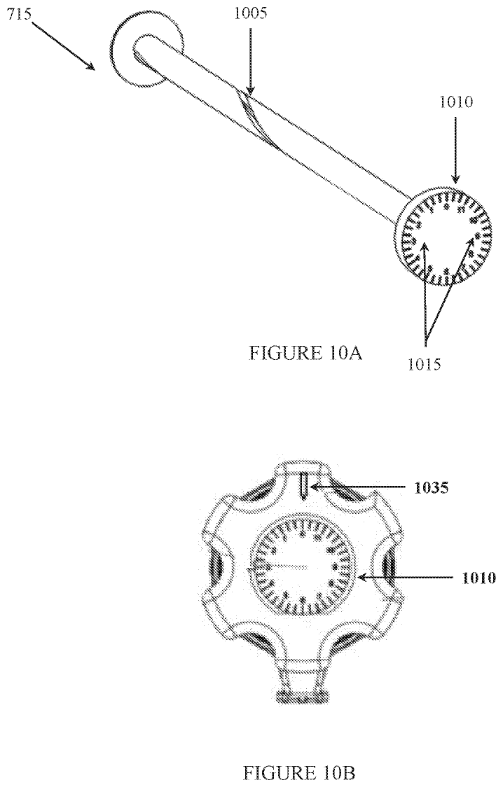

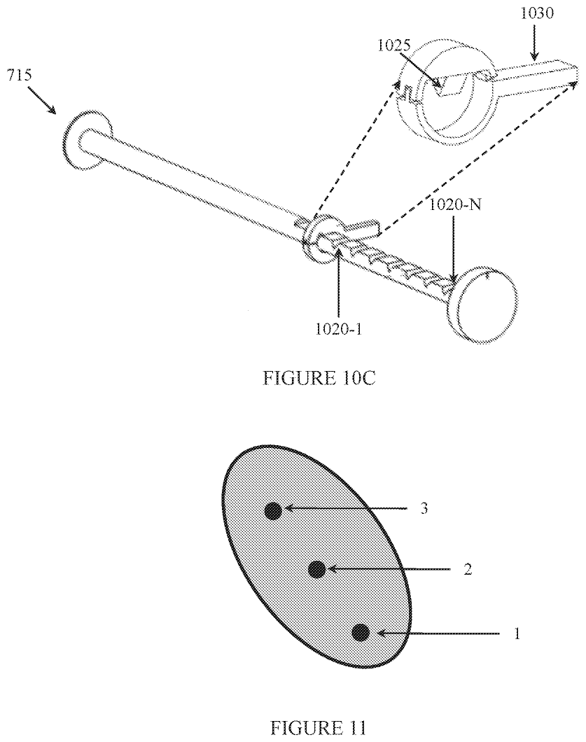

Referring to FIG. 7 and FIG. 8, in one embodiment of the present disclosure, the plunger 715 of the drug delivery device 115 is rotatably positioned inside the hollow cylindrical body 705 for delivering predetermined volume of the drug at the one or more locations in the target volume. FIG. 10A shows an exemplary plunger 715 in accordance with an embodiment of the present disclosure. As shown, the plunger 715 comprises a helical groove 1005 on the outer surface and a graduated knob 1010 at one end, the end near to the user. In one embodiment, the helical groove 1005 engages with a pin (not shown) on an inner surface of the hollow cylindrical body 705 for enabling a linear movement of the plunger 715 when the plunger 715 is rotated. That is, when the user rotates the graduated knob 1010 (the plunger 715), the plunger 715 moves linearly to push the drug stored in the hollow cylindrical body 705 of the drug delivery device 115, and hence to deliver the drug to the one or more locations in target volume. As described, the volume of drug to be delivered at each of the one or more locations in the target volume is dependent on the volume (size) of the target volume a shape of the target volume, location of the target volume in the predetermined volume, effectiveness of the drug, and the one or more locations. Further, as described, the rotation of the graduated knob 1010 of the plunger 715 moves the plunger 715 linearly to deliver the drug. In one embodiment of the present disclosure, the graduated knob 1010 comprises graduations 1015 for indicating a volume of the drug delivered when the plunger 715 is rotated for delivering the drug at the one or more locations in the target volume, wherein the volume of drug delivered is proportional to the angle of rotation of the knob 1010. It is to be noted that the volume of drug delivered depends on a length and an inner diameter of the hollow cylindrical body 705, and hence a person skilled in the art may design the drug delivery based on the requirements of drug delivery. For example, considering the hollow cylindrical body 705 having a length of 8 cm and radius of 1 cm, a 90° rotation of the plunger 715 delivers 3 ml of drug into the target volume. In one embodiment of the present disclosure, a reference marking is provided on the knob 805 of the barrel 120 to reset or adjust the graduated knob 1010 of the plunger for precise drug delivery. FIG. 10B shows the front view of the knob of the barrel with marking and graduated knob of the plunger in accordance with an embodiment of the present disclosure. The rotation of the plunger 715 will be measured with reference to the marking 1035. As described, the drug delivery device 115 disclosed in the present disclosure enables precise drug delivery at the one or more locations in the target volume.

FIG. 10C shows another exemplary plunger in accordance with an embodiment of the present disclosure. As shown, the plunger 715 is movable linearly inside the hollow cylindrical body 705 of the drug delivery device 115 when pushed from an end proximal to a user of the drug delivery device 115. In one embodiment, the plunger 715 comprises a plurality of grooves 1020-1 to 1020-N for accommodating a wedge 1025 of a restrictor 930 detachably clamped on to the plunger 715 for restricting a movement of the plunger 715 into the hollow cylindrical body 705, when the restrictor 930 comes in contact with the hollow cylindrical body 705. Each of the plurality of grooves 1020-1 to 1020-N define the pitch and the volume of drug when the plunger 715 is pushed. Further, each groove comprises markings for indicating a volume of the drug delivered when the plunger 715 is pushed for delivering the drug at the one or more locations in the target volume. Since the volume of the drug delivered is dependent on a length and an inner diameter of the hollow cylindrical body 705, a person skilled in the art may design the drug delivery based on the requirements of drug delivery, and the markings are provided accordingly to indicate the volume of drug delivered. Further, the number of grooves and a distance between the grooves may be designed as per the drug delivery requirement. During the use, the user may move the restrictor 1030 along the axis of the plunger 715 and clamp at any one of the grooves to define the volume of the drug to be delivered or to restrict further movement of the plunger 715 when the plunger 715 is pushed or for both the purposes. If the drug needs to be delivered at more than one location, the user may disengage the wedge 1025 partially by pressing the restrictor 1030 and may move the restrictor 1030 to the next defined position.

Having introduced the different elements of the system and the apparatus, a brief description of operation of the system 100 to deliver the drug to one or more locations in a target volume of a subject will now be described. It is to be noted that the disclosure is not concerned with the treatment of fibroids, tumors, cysts, and similar growths, but is concerned with the system and apparatus to deliver the drug to the one or more locations in the target volume for the treatment. The procedure is described here only to illustrate the operation and function of the elements of the system and apparatus, and how the system works. Hereafter, the target volume is considered as a fibroid for the ease of explanation and understanding.

During the drug delivery procedure, a subject (the patient) is anesthetized preferably with a local anesthetic to ensure that the patient does not feel the pain and discomfort associated with the procedure. Initially, while treating fibroids, the ultrasound transducer is inserted through an external orifice of the uterus so that the distal end of the transducer reaches the cervix. For the sake clarity, it is to be noted that the proximal end of the transducer has a cable for connecting to the ultrasound scanner 105 and the ultrasound transducer positioned on the cervix by the user. The distal end of the ultrasound transducer is then swept along the uterus to acquire a volume scan of the entire uterus (the predetermined volume) in different orientations. The resultant images are captured by the ultrasound scanner 105 and communicated to the computing device 110.

On receiving the images of the uterus from the ultrasound scanner 105, the computing device 110 displays the one or more images for annotation by the user. In one embodiment, the user may select the one or more images for annotations. Then the user may use one or more markers (colours, for example) to highlight the fibroids, the uterus, and other locations of the uterus, if required. On receiving the user annotations, the computation module 225 computes the navigational data to reach the fibroids from the orifice of the uterus. Further, the 3D model generator 220 generates a 3D model of the uterus. It is to be noted that the 3D model of the uterus is generated using a plurality of 2D images by any know methods. As described, a machine learning model is trained to identify the fibroids from the images of the uterus. The generated 3D model is displayed on the display module 130 for providing feedback to the user.

On identifying the fibroid, the computing device 110 computes the navigational data to reach the fibroid from an entry orifice of uterus. The navigational data include but not limited include but not limited to type of a sheath 305, length of insertion of the sheath 305 into the uterus, orientation of the sheath 305, length of the needle 710 to reach the one or more locations of the fibroid, and angles of rotation of a graduated knob 1010 of the drug delivery device 115 for delivering the drug at each of the one or more locations in the fibroid where the drug needs to be delivered. Further the computing device 110 determines a volume of the drug to be delivered to the one or more locations in the fibroid, based on the volume of the fibroid and the one or more locations in fibroid.

As described, the drug to be delivered depends on the concentration and effectiveness of the drug on the fibroid. For example, if the effective dose of a particular drug is 2 ml for one cubic centimetre of the fibroid, the computing device 110 considers the effectiveness of the drug on the fibroid, volume of the fibroid, and one or more determined locations, while recommending the volume of the drug to be delivered at the one or more locations. For example, for a fibroid of diameter of 7 cm, the target location defined may be the centre of the fibroid and 2 cm away from the centre on either side (Target locations 1,2 and 3), as shown in FIG. 11. Further, the computing device 110 computes the volume of drug to be delivered at each location and displays this information on the display module 130 for providing feedback to the user. For example, the volume of drug to be delivered at location 2 (centre) may be 3 ml and at location 1 and 3 may be 2 ml. So, the user operates the barrel 120 to move the needle 710 of the drug delivery device 115 to reach the farthest location (location 3 for example) from the entry orifice and then adjust and operate the plunger 715 of the drug delivery device 115 to precisely deliver the computed volume of the drug. Then the needle 710 is retracted to deliver the drug to the other locations, that is, location 2 and 3, for example. It is to be noted the drug is loaded into the hollow cylindrical body 705 of the drug delivery device 115 through the tip needle 710 by pulling the plunger 715 or by rotating the plunger 715 in anti-clockwise rotation.

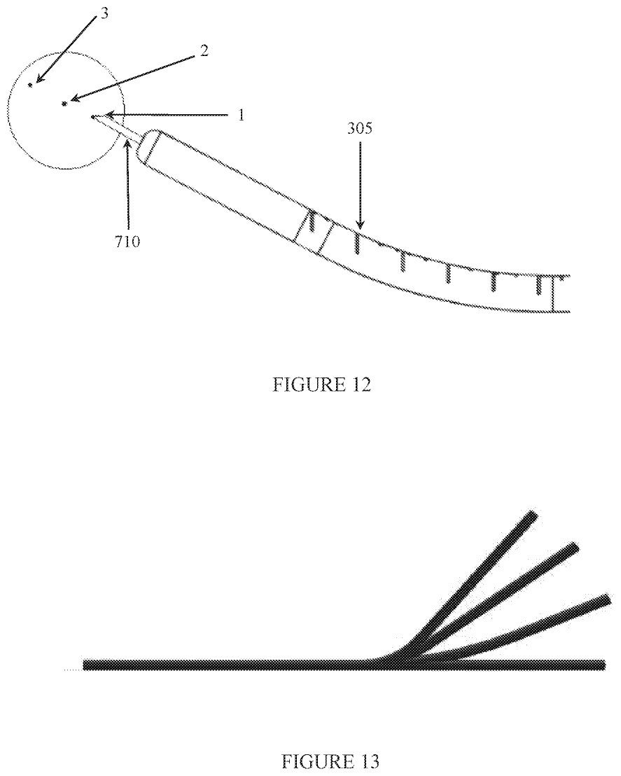

In one embodiment of the present disclosure, the system 100 is configured for providing real-time feedback to the user while performing the drug delivery procedure. That is, during the drug delivery procedure, the user may place an ultrasound transducer on the abdomen of the subject such that the transducer scans both the fibroid and the angulated tip of the needle. The video captured by the ultrasound scanner 105 is fed to the computing device 110. Referring to FIG. 2, in one embodiment of the present disclosure, the feedback module 230 of the computing device 110 arguments the 3D model on the real-time image for providing real-time feedback to the user. FIG. 12 shows exemplary feedback displayed by the computing device in accordance with an embodiment of the present disclosure.

As described, the system disclosed in the present disclosure determines the one or more locations in the target volume is determined based on at least one of a volume of the target volume, a shape of the target volume, and a location of the target volume within the predetermined volume. Further, the system determines the navigational data to reach the one or more location in the target volume, the navigational data including but not limited to, a type of the sheath, a length of insertion of the sheath into the pre-determined volume, an orientation of the sheath, a length of the needle to reach the one or more locations of the target volume, and angles of rotation of a graduated knob for delivering the drug at each of the one or more locations in the target volume. Furthermore, the system computes a volume of the drug to be delivered to the one or more locations of the target volume. On determining all the data and associated values, the system displays the same on the display module for assisting the user while the drug delivery procedure.

It is to be noted that the drug delivery device 115 disclosed in the present disclosure may be used with any known systems. However, the use of the drug delivery device 115 with the system 100 enables precise delivery of the drug to the one or more intended location in a target volume, and hence enhances the effectiveness of the treatment. Further, the drug delivery device 115 disclosed in the present disclosure is designed to be held in hand and mountable on a support structure.

In other embodiments drug targeting is improved in drug delivery systems with the help of novel drug loaded inserts, hydro gel systems containing phase change polymers which can have transition in response to body temperature, liposomes, micro emulsion-based drugs, and nanoparticles which can direct the drug to its site of action. Employment of nanoparticles provides massive advantages regarding drug targeting, delivery, and release. Nanoparticles are polymeric particles made of natural or artificial polymers ranging in size between about 10 and 1000 nm (1 mm). Example Poly (butyl cyanoacrylate) nanoparticles.

In one embodiment of the present disclosure, the system 100 disclosed in the present disclosure may be used for biopsy, by replacing the drug delivery device 115 by a biopsy needle. It is to be noted that the biopsy needle may be used with or without sheath. However, the use of sheath provides feedback on the orientation of the device and acts as a cannula for guiding the biopsy needle. It is to be noted that the system disclosed in the present disclosure is also intended to be used for biopsy of fibroids before their ablation or excision or drug delivery. This helps to potentially avoid a surgery and provide a definitive way of diagnosis.

Further, as described in the present disclosure, the sheath may have different bent profiles to reach any target volume of a subject. In one embodiment, the sheath comprises multiple detachable profiles tips. FIG. 13 shows sheaths with different bent profiles.

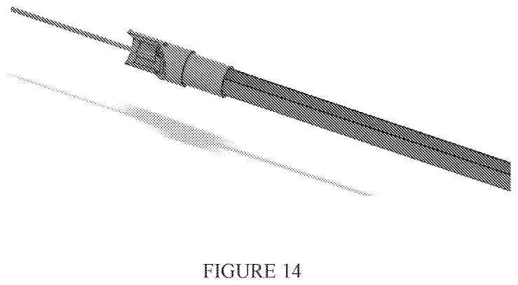

In one embodiment of the present disclosure, the sheath includes profiles with belt movement or gear mechanism. FIG. 14 shows an exemplary pivotable sheath with pivotable profiles in accordance with an embodiment of the present disclosure. Further, shape memory alloys with controlled shape adjustments or distal tip with adjustable angles may be used. It is to be noted that the multiple bent profiles and distal tips attachments distal tips may be differentiated using colour coding, labelling, unique shapes, unique visible identifications, or any know methods.

The system and device disclosed in the present disclosure may be used for delivering a drug precisely to the intended (system determined) region in a target volume with the help of 3D model of a predetermined volume, the drug delivery device, and the feedback features of the drug delivery device and the system.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

1. A system for delivering a drug into a target volume of a subject, the system comprising:
an ultrasound scanner configured for performing a volume scan of a predetermined volume of the subject, wherein the scanned predetermined volume includes the target volume;
a computing device, communicatively connected to the ultrasound scanner, the computing device configured for at least generating a three-dimensional model of the predetermined volume and computing navigational data to reach the target volume from an entry orifice of the volume of the subject;
a housing unit, for accommodating a barrel, to which a sheath is detachably attached; and
a drug delivery device for delivering the drug at one or more locations in the target volume, wherein the drug delivery device comprises:
a hollow cylindrical body comprising a needle at a first end, the hollow cylindrical body configured for storing the drug to be delivered and configured for moving the needle linearly through the sheath to pierce the target volume and to reach the one or more locations in the target volume; and
a plunger, positioned inside the hollow cylindrical body, wherein the plunger is configured for enabling delivery of a predetermined volume of a drug to the one or more locations in the target volume.

2. The system as claimed in claim 1, wherein the hollow cylindrical body of the drug delivery device comprises a helical groove on an outer surface of the hollow cylindrical body, wherein the helical groove engages with a pin on an inner surface of the barrel, and a rotation of the barrel moves the drug delivery device linearly for moving the needle of the drug delivery device, through the sheath, for piercing the target volume and to reach the one or more locations inside the target volume.

3. The system as claimed in claim 1, wherein the housing unit comprises markings for indicating the length of movement of the needle when the barrel is rotated to move the drug delivery device.

4. The system as claimed in claim 1, wherein the housing unit comprises a barrel locking pin for preventing the transitional movement and for allowing the rotational movement of the barrel with reference to the housing unit.

5. The system as claimed in claim 1, wherein the plunger of the drug delivery device is rotatably positioned inside the hollow cylindrical body for delivering a predetermined volume of the drug at the one or more locations in the target volume.

6. The system as claimed in claim 1, wherein the plunger comprises:
a helical groove on the outer surface of the plunger, wherein the helical groove engages with a pin on an inner surface of the hollow cylindrical body for enabling a linear movement of the plunger when the plunger is rotated; and
graduated a knob at one end of the plunger, for rotating the plunger, the graduated knob having graduations for indicating a volume of the drug delivered when the plunger is rotated for delivering the drug at the one or more locations in the target volume, wherein the volume of drug delivered is proportional to the angle of rotation of the graduated knob.

7. The system as claimed in claim 1, wherein the plunger is linearly movable inside the hollow cylindrical body when pushed from an end proximal to a user of the drug delivery device.

8. The plunger as claimed in claim 6, wherein the plunger comprises a plurality of grooves for accommodating a wedge of a restrictor detachable clamped on to the plunger for restricting a movement of the plunger into the hollow cylindrical body, when the restrictor comes in contact with the hollow cylindrical body.

9. The system as claimed in claim 1, wherein the drug delivery device is for delivering the drug at the one or more locations in a fibroid and the computing device is configured for generating a 3D model of a uterus of the subject and computing navigational data to reach the fibroid from an external orifice of the uterus.

10. The system as claimed in claim 1, wherein the one or more locations in the target volume is determined based on at least one of a volume of the target volume, a shape of the target volume, and a location of the target volume within the predetermined volume.

11. The system as claimed in claim 1, wherein the navigational data includes, type of the sheath, a length of insertion of the sheath into the pre-determined volume, an orientation of the sheath, a length of the needle to reach the one or more locations of the target volume, and angles of rotation of a graduated knob for delivering the drug at each of the one or more locations in the target volume.

12. The system as claimed in claim 1, wherein the computing device is configured for determining, based at least one of a volume of the target volume, a shape of the target volume, location of the target volume in the predetermined volume, effectiveness of the drug, a volume of the drug to be delivered to the one or more locations of the target volume.

13. The system as claimed in claim 1, wherein the needle is a flexible needle and is movable through the sheath that is bent at a predefined angle for reaching the one or more locations in the target volume
